(19) United States
(12) Patent Application Publication
(10) Pub. No.: US 2008/0009710 A1
(43) Pub. Date: Jan. 10, 2008

(54) Magnetic Resonance Imaging Method and Apparatus

(76) Inventors: Hiroyuki Itagaki, Tokyo (JP); Tetsuhiko Takahashi, Tokyo (JP)

Correspondence Address: COOPER & DUNHAM, LLP, 1185 AVENUE OF THE AMERICAS, NEW YORK, NY, 10036, US

(21) Appl. No.: 11/883,700

(22) PCT Filed: Feb. 7, 2006
(86) PCT No.: PCT/JP06/02012

§ 371 Date: Aug. 2, 2007

(30) Foreign Application Priority Data
Feb. 16, 2005 (JP) 2005-038708

(51) Int. Cl. A61B 5/055

(52) U.S. Cl. 600/419

(57) ABSTRACT

A magnetic resonance imaging method includes a step (1) for exciting atomic nuclei in a desired region of an object to be examined so as to cause nuclear magnetic resonance, a step (2) for detecting a nuclear magnetic resonance signal generated in the blood, and a step (3) for extracting a blood image of the object by the detected nuclear magnetic resonance signal. The desired region excited by the step (1) represents a plurality of regions arranged at a predetermined interval.

### TECHNICAL FIELD

The present invention relates to a magnetic resonance imaging method (hereinafter referred to as an MRI method) and apparatus, in particular to an MRI method and apparatus capable of generating high-quality images of blood stream.

### BACKGROUND ART

In Arterial Spin Labeling (hereinafter referred to as ASL) which is a technique for exciting in advance a desired region of an object to be examined and imaging a blood stream in downstream of the excited region, a method is disclosed in Non-Patent Document 1 for imaging by conforming direction of gradient magnetic field for slice selection and gradient magnetic field for readout.

Non-Patent Document 1: W. G. Rehwald et al.: GCFP-A New Non-Invasive Non-Contrast Cine Angiography Technique Using Selective Excitation and Global Coherent: Proc. Intl. Soc. Mag. Reson. Med. 11 (2004)

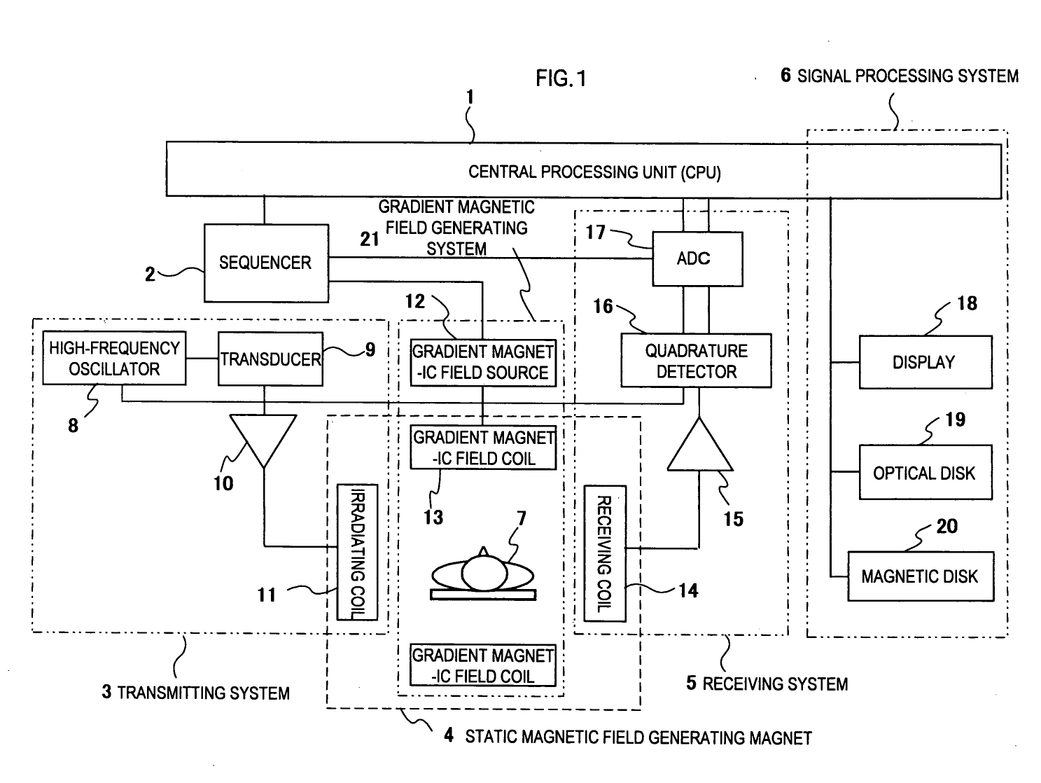

With usage of the imaging method disclosed in Non-Patent Document 1, an excited plane excited by slice selection and an imaging area imaged by accumulating NMR signals intersect orthogonally, whereby making it possible to generate an image of blood vessel extended from the excited plane. In Non-Patent Document 1, images of a blood vessel extended from an excited plane over time are illustrated particularly in FIG. 1.

However, the following problem still remains in the conventional technique disclosed in Non-Patent Document 1. That is, while the regions excited by slicing selection are only excited planes in the conventional technique disclosed in Non-Patent Document 1, the signals from blood flowed out of the excited plane attenuates its intensity over time due to relaxation phenomenon. Thus lowering performance for blood vessel description positioned apart from the excited plane remains as a problem.

Also in Non-Patent Document 1, an imaging sequence that is simulating SSFP for collecting NMR signals is used. In other words, NMR signals are collected while continuously applying a plurality of RF pulses having small flip angles. However, the imaging method by such imaging sequence has a tendency of being influenced by turbulence of signal phase. For this reason, deterioration of images occurs in regions where the static magnetic field is not uniform or in regions having high velocity of blood flow. Such problems are not taken into consideration in the method disclosed in the above-mentioned document.

### DISCLOSURE OF THE INVENTION

The objective of the present invention is to provide an MRI method and apparatus capable of imaging a wide range of blood flow with high quality image using the ASL method.

In order to achieve the above-mentioned objective, an MRI method of the present invention comprises:
a step (1) for exciting atomic nuclei in a desired region of an object to be examined so as to cause nuclear magnetic resonance;
a step (2) for detecting nuclear magnetic resonance signals generated in the blood; and
a step (3) for extracting an image of blood of the object by the detected nuclear magnetic resonance signals,
wherein the desired region excited by the step (1) represents a plurality of regions arranged at a predetermined interval.

Also, the MRI apparatus of the present invention comprises:
static magnetic field generating means for generating a static magnetic field in an imaging space where an object to be examined is placed;
gradient magnetic field generating means for generating a gradient magnetic field in the imaging space;
high-frequency magnetic field generating means for generating a high-frequency magnetic field to cause nuclear magnetic resonance to the object in the imaging space;
signal receiving means for detecting nuclear magnetic resonance from the object;
signal processing means for reconstructing an image using the detected nuclear magnetic resonance signals;
measurement control means for controlling the gradient magnetic field generating means, high-frequency magnetic field generating means and signal processing means based on a predetermined pulse sequence; and
display means for displaying the image,
wherein the measurement control means comprises means for controlling application of high-frequency magnetic field by the high-frequency magnetic field generating means so as to excite the plurality of excited regions arranged in the predetermined interval in the body of the object.

### BRIEF DESCRIPTION OF THE DIAGRAMS

FIG. 1 is a diagram showing a system configuration of an MRI apparatus related to the present invention.

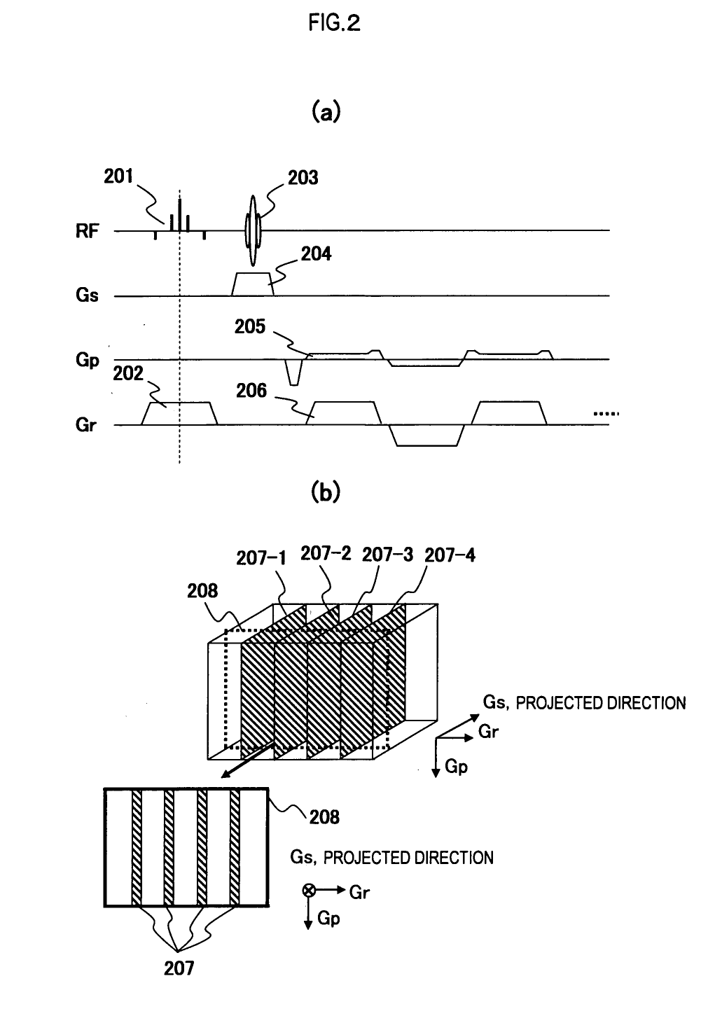

FIG. 2 (a) is a diagram showing an imaging sequence of the MRI method related to embodiment 1, and (b) is a diagram showing the correspondence between excited planes and an imaging area upon executing imaging sequence related to embodiment 1.

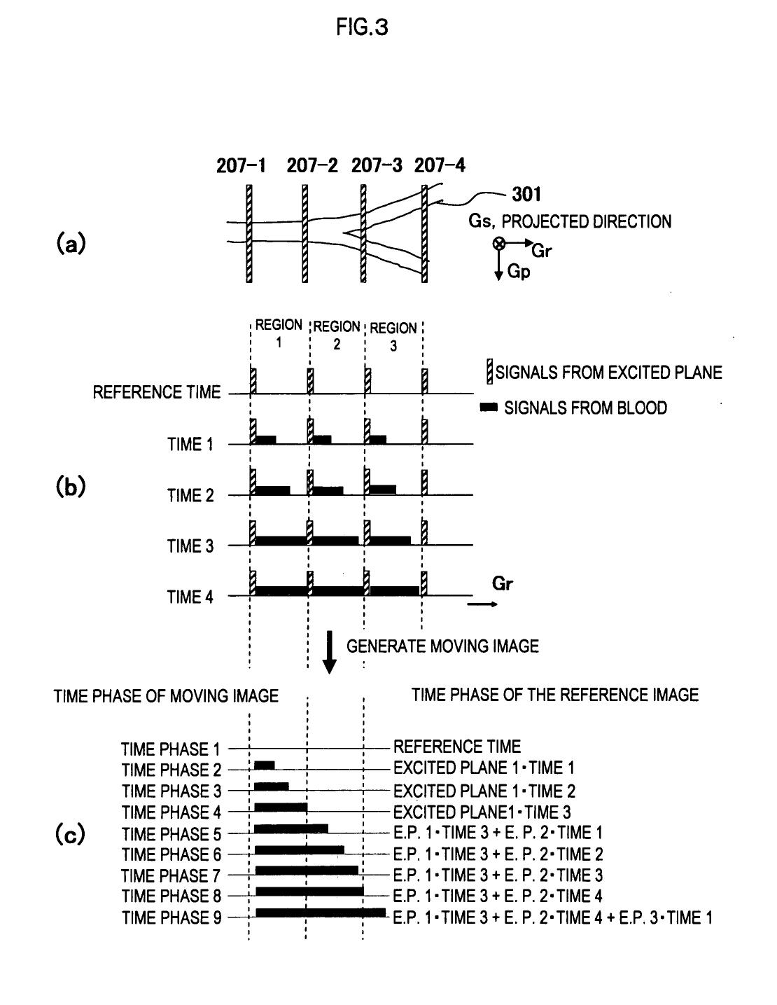

FIG. 3 (a) is a diagram showing a blood vessel targeted for image extraction and a plurality of excited planes, and (b) is a diagram showing to what extent the downstream of the blood flow from the excited plane is extracted by the images generated after each time passage after applying RF burst pulse 501, and (c) is a diagram showing a moving image generated to present the blood flowing continuously from a specific excited plane.

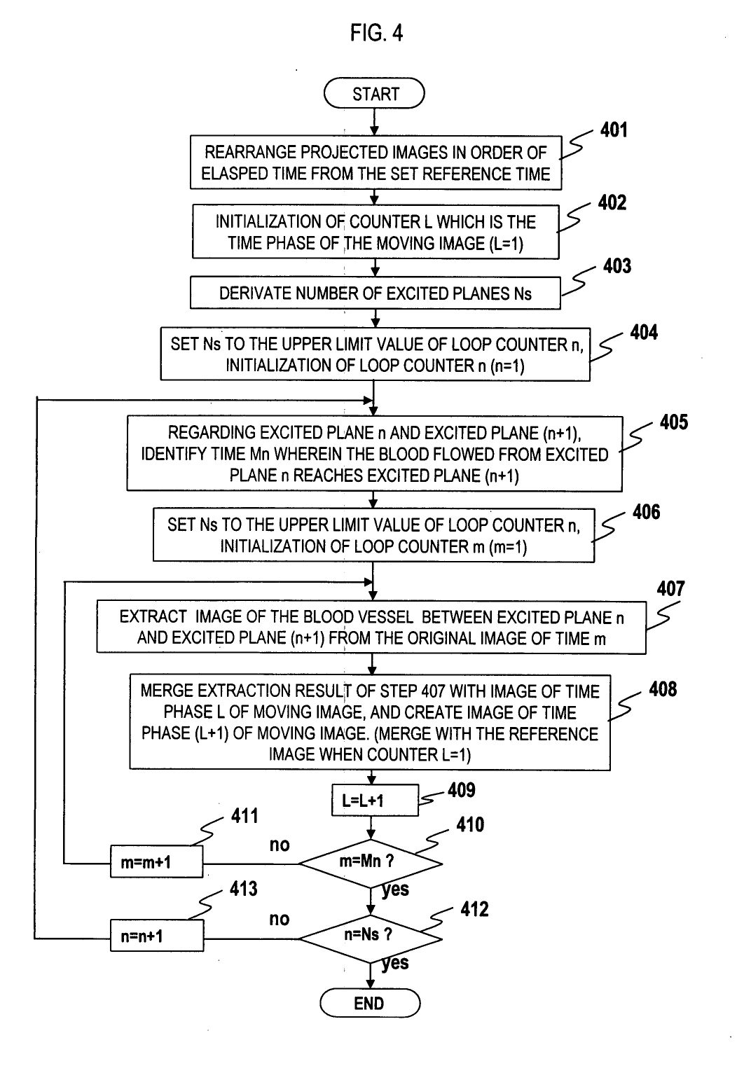

FIG. 4 is a flow chart showing concrete procedure for generating moving images.

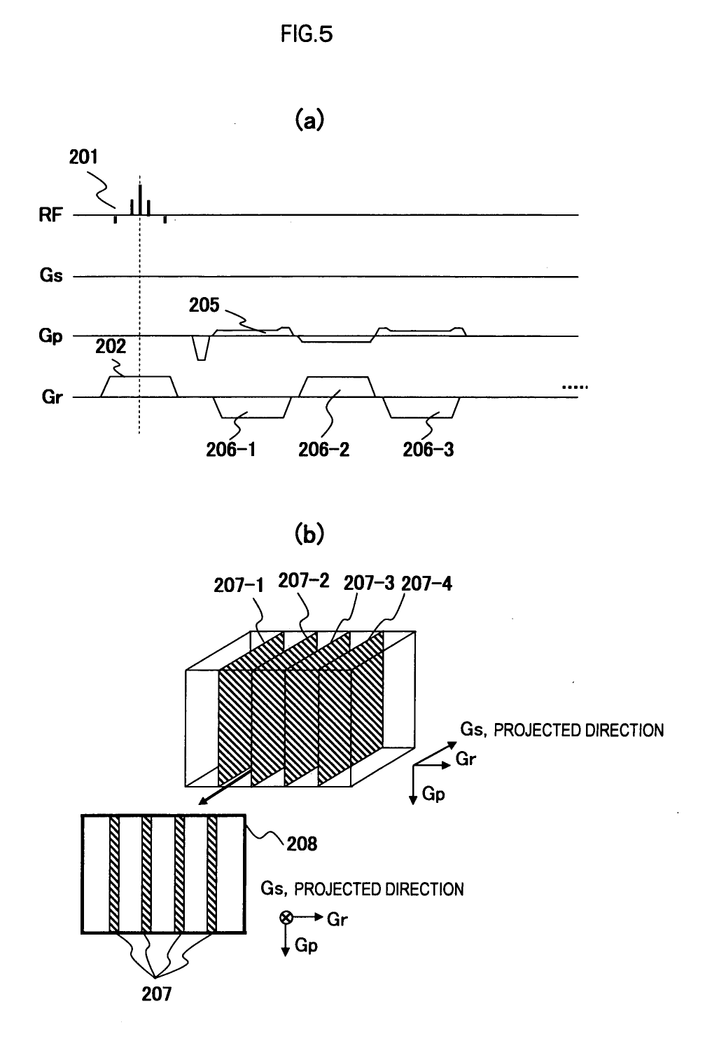

FIG. 5 (a) is a diagram showing an imaging sequence of an MRI method related to embodiment 2, and (b) is a diagram showing the correspondence between the excited planes and the imaging area upon executing the imaging sequence related to embodiment 2.

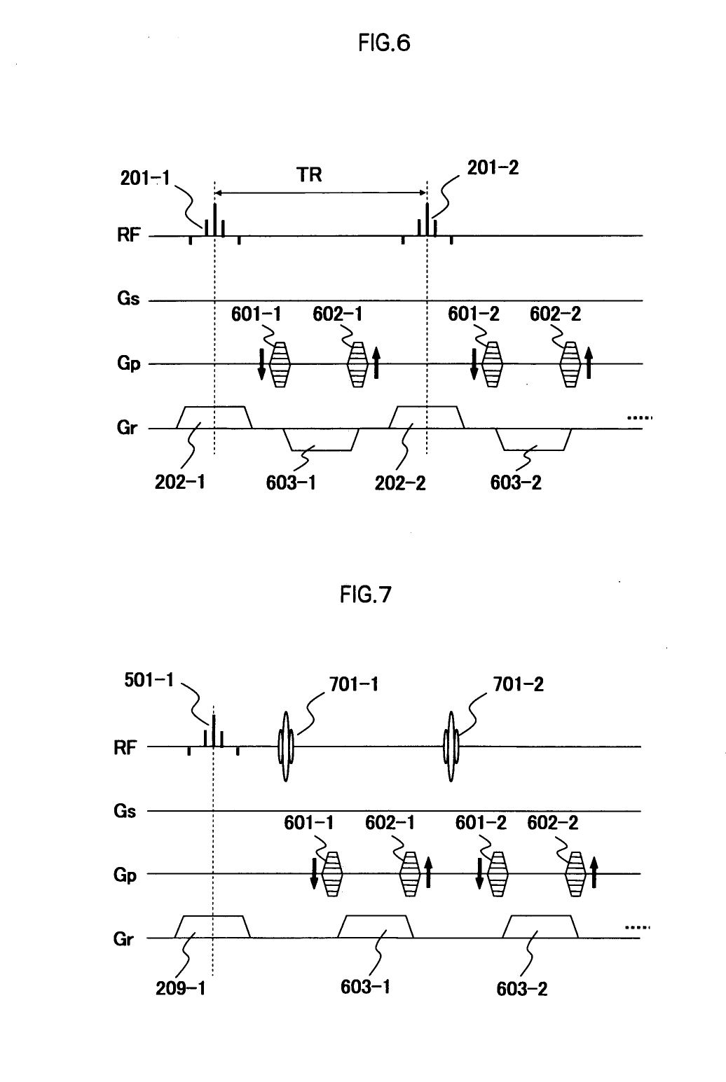

FIG. 6 is an imaging sequence diagram of an MRI method related to embodiment 3.

FIG. 7 is an imaging sequence diagram of an MRI method related to embodiment 4.

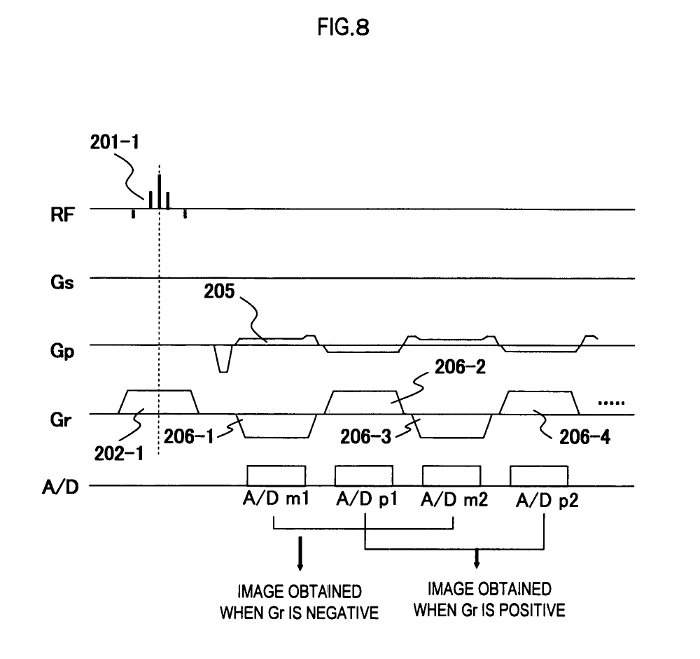

FIG. 8 is an imaging sequence diagram of an MRI method related to embodiment 5.

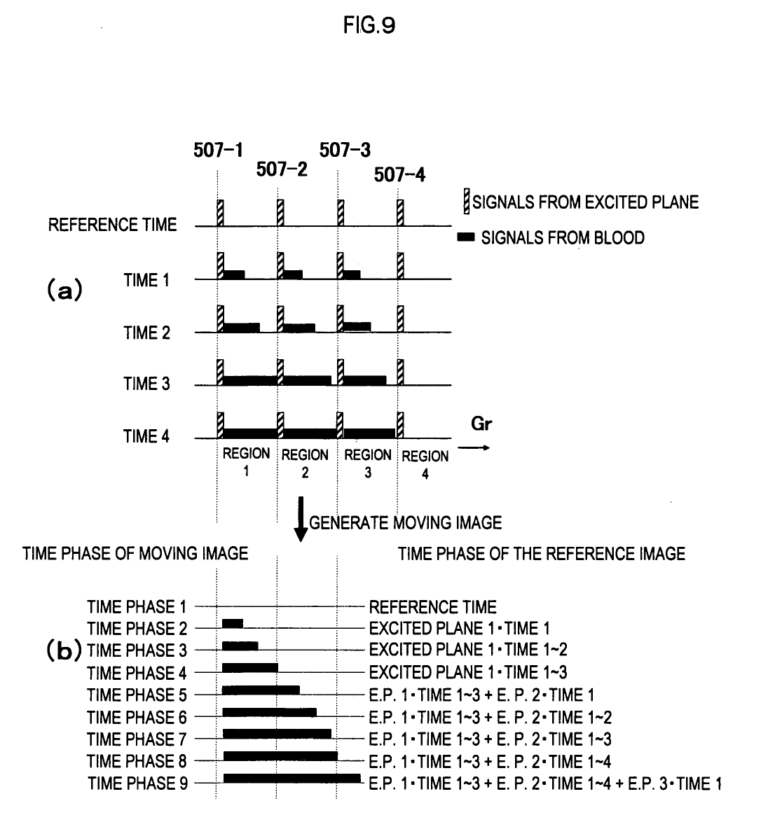

FIG. 9 (a) is a diagram showing to what extent the downstream of the blood stream from the excited plane is extracted by the images generated after each passage of time since the first application of RF burst pulse 501, and (b) is a diagram showing generation of a moving image in embodiment 6.

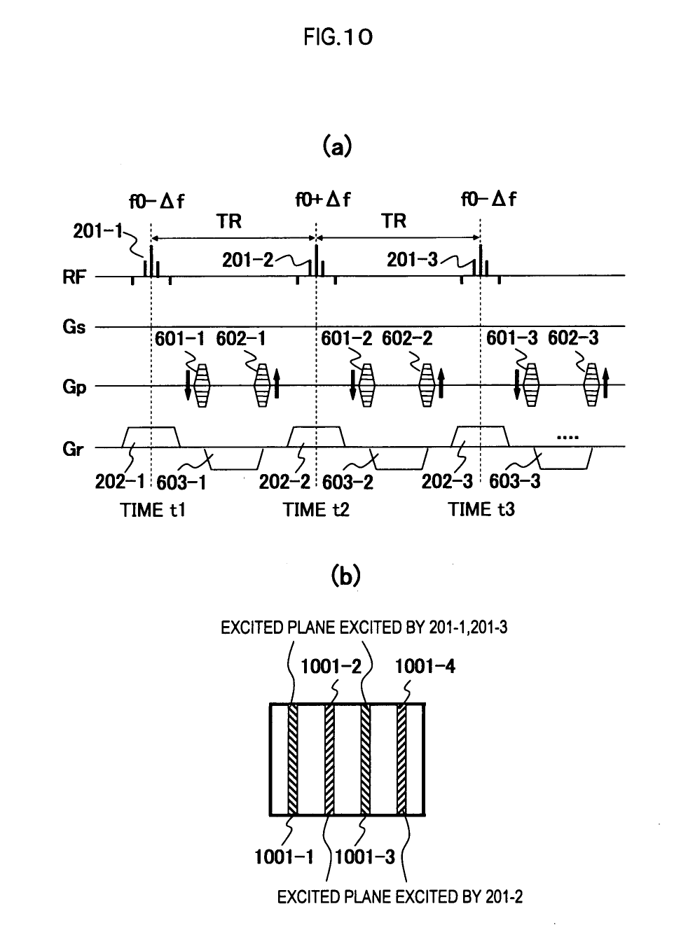

FIG. 10 is an imaging sequence diagram of an MRI method related to embodiment 7, and (b) is a diagram showing how the excited planes are excited by two kinds of burst RF pulses.

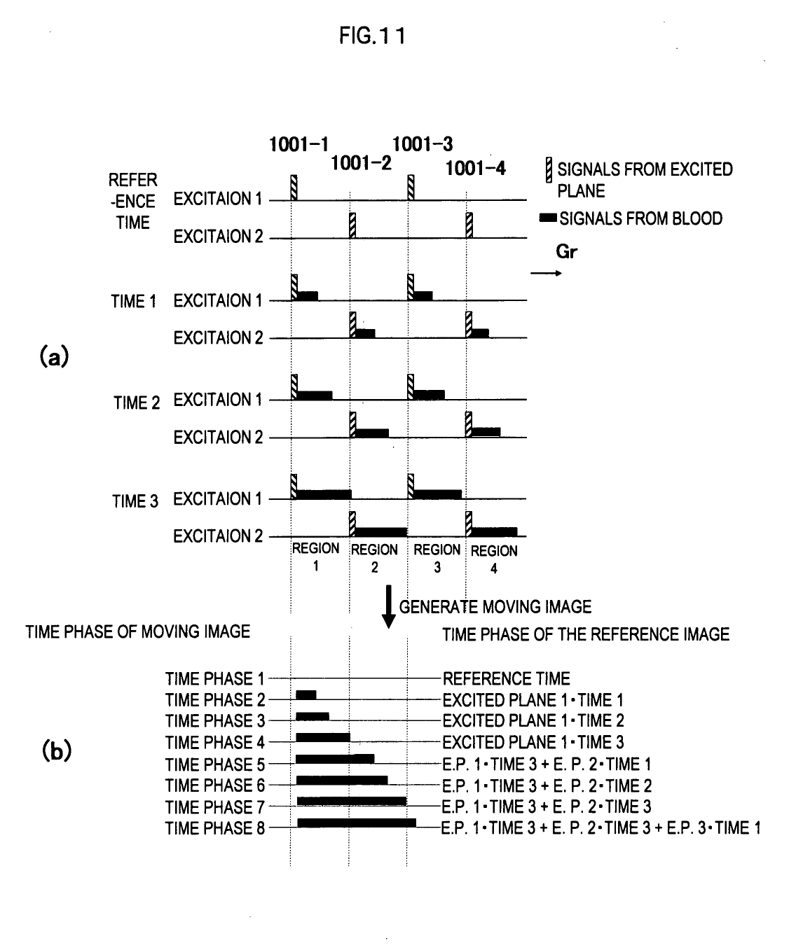

FIG. 11 (a) is a diagram showing to what extent the downstream of a blood stream is extracted by the images generated after each passage of time from the reference time, and (b) is a diagram showing a moving image generated in embodiment 7.

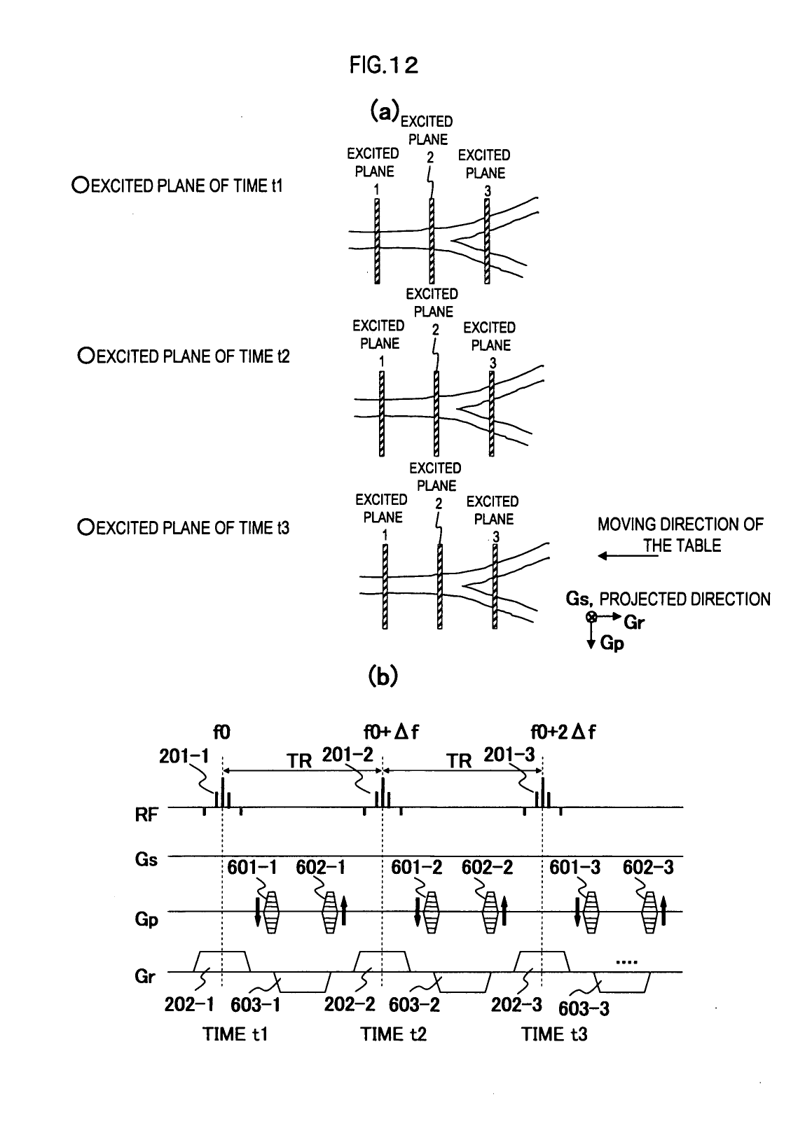

FIG. 12 (a) is a diagram showing how an object and the excited planes should be moved in embodiment 8, and (b) is an imaging sequence diagram of an MRI method related to embodiment 8.

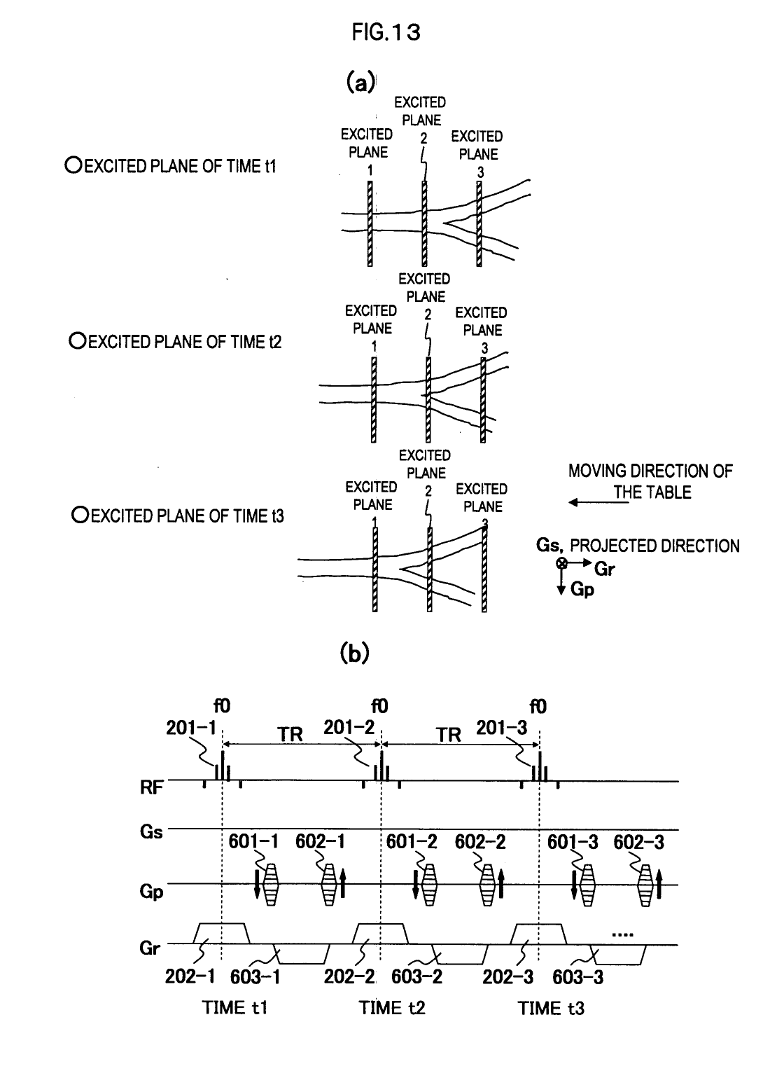

FIG. 13 (a) is a diagram showing how an object and the excited planes should be moved in embodiment 9, and (b) is an imaging sequence diagram of an MRI method related to embodiment 9.

### BEST MODE FOR CARRYING OUT THE INVENTION

Hereinafter, system configuration of an MRI apparatus related to the present invention will be described in detail referring to FIG. 1.

Configuration of the MRI apparatus is classified broadly by central processing unit (hereinafter referred to as CPU) 1, sequencer 2, transmitting system 3, static magnetic field generating magnet 4, receiving system 5, gradient magnetic field generating system 21, and signal processing system 6.

CPU 1 controls sequencer 2, transmitting system 3, receiving system 5 and signal processing system 6 according to the program set in advance. Sequencer 2 is operated based on control commands from CPU 1, and transmits various commands necessary for collecting image data for generating tomographic images of object 7 to transmitting system 3, gradient magnetic field generating system 21 and receiving system 5.

Transmitting system 3 comprises devices such as high-frequency oscillator 8, modulator 9, irradiating coil 11 and RF shield, amplitude-modifies the reference high-frequency pulse from high-frequency oscillator 8 by the command of sequencer 2, and irradiates a predetermined pulsed electromagnetic waves to the object by amplifying the amplitude-modulated high-frequency pulse via high-frequency amplifier 10 and providing it to irradiating coil 11.

Static magnetic field generating magnet 4 generates a homogeneous static magnetic field around object 7 in a predetermined direction. Inside of static magnetic field generating magnet 4, irradiating coil 11, gradient magnetic field coil 13 and receiving coil 14 are disposed. Gradient magnetic field coil 13 is included in gradient magnetic field generating system 21, receives provision of current from gradient magnetic field source 12, and generates gradient magnetic field under the control of sequencer 2.

Receiving system 5 is for detecting NMR signals emitted by nuclear magnetic resonance of atomic nuclei in the body of the object, and has receiving coil 14, amplifier 15, quadrature detector 16 and A/D converter 17. NMR signals as a response of the object to the electromagnetic waves irradiated from the above-mentioned irradiating coil 11 are detected in receiving coil 14 disposed in the vicinity of the object, inputted to A/D converter via amplifier 15 and quadrature detector 16, and converted into digital quantity. Then the signals converted into digital quantity are transmitted to CPU 1.

Signal processing system 6 comprises an external memory device such as magnetic disk 20 and optical disk 19, and display 18 formed by devices such as CRT. When data from receiving system 5 is inputted, CPU 1 performs process such as signal processing and image reconstruction. Images of the desired fault plane of object 7 which are the result of the above-mentioned process are displayed on display 18, and stored in an external memory device such as magnetic disk 20.

### EMBODIMENT 1

First, an imaging sequence of an MRI method related to embodiment 1 will be described in order using FIG. 2 (a). The present embodiment is formed by an image acquisition step for collecting image data by the imaging sequence shown in FIG. 2 (a), and an image composition step for generating moving images for presenting blood flow flowing from upstream over time based on the image data obtained by the image acquisition step. Hereinafter, the image acquisition step will be described referring to FIG. 2, and the image composition step will be described referring to FIGS. 3 and 4.

FIG. 2 (a) is an image-sequence diagram showing an image-acquisition step in the present embodiment. In FIG. 2 (a), RF represents a line indicating application of high-frequency magnetic field pulse (RF pulse), Gs represents a line indicating application of gradient magnetic field for slice selection, Gp represents a line indicating application of phase-encode gradient magnetic field, and Gr represents a line indicating application of gradient magnetic field for readout 201 indicates pulses for exciting a plurality of excited planes (207-1˜207-4 in FIG. 2 (b)) that are mutually disposed at intervals simultaneously, and is generally referred to as a burst RF pulse. Burst RF pulse 201 is an RF pulse wherein a plurality of unit RF pulses of short time duration are combined, amplitude of the plurality of unit RF pulses is amplitude-modulated in the form of function as a whole, and time interval of the respective unit RF pulses is set corresponding to the interval of the excited planes (refer to Non-Patent Document 2 for an example of burst RF pulses).

Non-Patent Document 2: H. Ochi et al.: Dual-frequency amplitude-modulated BURST Imaging, International Society for Magnetic Resonance in Medicine, 5^{th} Scientific Meeting and Exhibition P1824(1997).

Also, 202 indicates application of a gradient magnetic field pulse for slice selection applied along with the application of burst RF pulse 201, and it is to be applied in the same direction as direction of the gradient magnetic field pulse for readout (Gr direction). 203 indicates an inverting pulse (inverting high-frequency magnetic field pulse; π pulse), 204 indicates gradient magnetic field pulse 204 in Gs direction to be applied with inverting pulse 203 and these pulses are for inverting magnetization of the selected region (the region indicated by 208 in FIG. 2 (b)). Further, 205 and 206 indicated on lines Gp and Gr represents gradient magnetic field pulse for phase encoding and gradient magnetic field pulse for readout for continuously obtaining a plurality of signals by respectively inverting polar character of the gradient magnetic field pulse like an EPI sequence.

Next, FIG. 2 (b) is a schematic view illustrating the correspondence between the excited plane and the imaging area upon executing the imaging sequence related to embodiment 1.

In FIG. 2 (b), 207-1˜207-4 indicate four excited planes excited by burst RF pulses, 208 indicates an imaging area slice-selected by inverting pulse 203 and gradient magnetic field pulse 204, and the lower diagram of FIG. 2 (b) indicates a cross-section being cut by the imaging area. These diagrams show that the excited planes and non-excited planes are being arranged alternately.

In the present embodiment, after the plurality of excited planes disposed mutually at intervals are excited simultaneously, the signals are collected, and images in each elapsed time are reconstructed from the first application of a burst RF pulse. In this way, in the present embodiment, it is possible to perform imaging of the blood flow with passage of time. The above-mentioned image data in each elapsed time obtained by the imaging sequence shown in FIG. 2 (a) is stored, for example, in magnetic disk 20 and read out by CPU to be processed at each elapsed time in an image composition step which will be described below.

Next, the detail of the imaging composition step will be described referring to FIG. 3, which is for performing signal processing and generation of moving images using a plurality of echo signals obtained by the imaging sequence shown in FIG. 2 and imaging the state of blood flowing from the excited plane over passage of time.

FIG. 3 (a) shows a blood vessel as an imaging target, and a plurality of excited planes. 301 is the blood vessel, and 207-1˜207-4 are the plurality of excited planes excited by burst RF pulse 201. Next, FIG. 3 (b) shows, in the images generated at each passage of time, to what extent the downstream the blood is flowing from the excited plane.

By the above-mentioned diagram, while the image of the blood flowing from of the excited plane is not extracted, it can be recognized that the extracted region of the blood flow is extending from the excited plane to a location downstream as the time passes from time 1 to time 4. However, the blood flow extracted in the image (the blood vessel image) in time 1 time 4 which are generated in the regions between the respective excited planes (the region between excited planes 207-1 and 207-2 is region 1, the region between excited planes 207-2 and 207-3 is region 2 and the region between excited planes 207-3 and 207-4 is region 3) are discrete, since the blood flow is extracted in the image from each of excited plane 207-1˜excited plane 207-4. Given this factor, generation of moving images is executed in the present embodiment for making the image look like the blood is continuously flowing from a specific excited plane (from excited plane 207-1 here) by the method shown in FIG. 3 (c).

The moving image in FIG. 3 (c) is formed by time phase 1˜time phase 9. In the moving image at each time phase, the state of blood flow is extracted from upstream part of the region along with elapsed time.

The image is created by combining the blood extraction image of the target region in each time phase and the blood extraction image at the time when the blood reaches the utmost downstream side in the upstream side region with reference to the targeted region. More specifically, first in time phase 1, a moving image is generated using an image as it is in the reference time. In time phase 2, region 1 of the moving image is generated using the blood flow of time 1 in FIG. 3 (b). In time phase 3, region 1 of the moving image is generated using the blood flow of time 2 in FIG. 3 (b). In time phase 4, region 1 of the moving image is generated using the blood flow of time 3 in FIG. 3 (b).

Next, in time phase 5, the moving image of region 1 is generated using the blood flow of time phase 3, and the moving image of region 2 is generated using the blood flow of time phase 1 in FIG. 3 (b). Hereinafter, the moving images in time phase 6˜9 are generated in the same manner. By generating moving images as mentioned above, it is possible to extract a blood vessel as if the blood is flowing from a specified excited plane (excited plane 207-1 here). In the above-mentioned composition method of moving images, the time that blood reaches from excited plane 207-1 to excited plane 207-2 in region 1 is obtained as time 3, and the time that the blood reaches from excited plane 207-2 to excited plane 207-3 is obtained as time 4. Then upon generating the moving image in the downstream region, the moving image should be synthesized using the image of the time when the blood flow reaches the utmost of downstream (time 3 in region 1 and time 4 in region 2) in the upstream region. By such method, connection of the blood flow between the respective regions becomes smooth.

Next, concrete procedure of the image composition step for generating the moving image in FIG. 3 (c) from image data of FIG. 3 (b) will be described using a flow chart shown in FIG. 4. The program for executing the procedure described below will be stored in magnetic disk 20, and will be executed by being read out to CPU 1 as the need arises.

(Step 401)

According to the time passed from the previously set reference time (elapsed time), the obtained images are rearranged. The reference time is the time when, for example, the first burst RF pulse 201 is applied, and the image generated by collecting echo signals immediately after the reference time is set as the reference image.

{Step 402}

Counter related to the time phase of the moving image is set as L, and the default value thereof is set as 1.

(Step 403)

Position and number of the excited planes which are necessary parameter in the step described below are derived based on data (imaging condition) such as how burst RF pulses or gradient magnetic field pulses are applied for collecting image data. Or, they also can be calculated based on imaging data. In concrete terms, for example, a threshold value of signal intensity is set in image data, the region having the signal intensity more than the threshold value is detected as the excited plane, and the position and number of the excited plane thereof is calculated. And the excited plane at the utmost upstream point is set as the reference excited plane.

(Step 404)

Counter related to the excited plane is set as n, the default value thereof is set as 1, and the upper limit value is set as Ns being obtained in (step 403).

(Step 405)

Attention is paid on region n which is sandwiched between excited plane n and excited plane (n+1), and time Mn which is the time that the signals of blood that has flowed from excited plane n reaches excited plane (n+1) is identified. Concretely, identification of time Mn is defined by, for example, setting a threshold value to the signal intensity with respect to the pixel which is positioned on the side of excited plane n and adjacent to excited plane (n+1), and defining the time which is more than the threshold value as the time that the blood that has flowed from excited plane n reaches excited plane (n+1) (in the case of an example illustrated in FIG. 4, Mn(M₁) corresponding to region 1 is time 3, and Mn(M₂) corresponding to region 2 is time 4). Derivation of Mn in the present step is carried out while imaging data stored in magnetic disk 20 is being read out one item at a time. In the present step, derivation of Mn is calculated in all of region n.

(Step 406)

The counter related to elapsed time from the reference time for being used upon generation of a moving image of blood flow in the respective regions is set as m. Here, default value of counter m is set as 1, and maximum value of counter m upon extraction of region n is set as Mn.

(Step 407)

A blood vessel image is extracted with respect to region n being sandwiched between excited plane n and excited plane (n+1), from the image after elapsed time m from the reference time. Extraction of a blood vessel image in the present invention is carried out while imaging data is being read out from magnetic disk 20 to CPU 1, and the data of the extracted blood vessel is stored in magnetic disk 20 for the time being.

(Step 408)

The blood vessel image extracted in (step 407) and the moving image at time phase L are synthesized, and set as the moving image at time phase L+1. At that time, the blood vessel image extracted in (step 407) and image data in region 1˜region n−1 (only region 1 in the case that n=2) at time phase L are synthesized. In this regard, however, when the blood vessel image at region 1 is extracted in (step 407), the extracted image is used as it is at time phase L+1. Image composition of moving images in the present step is carried out while the blood vessel image extracted in (step 407) and stored in magnetic disk 20 and the blood vessel image at time phase L are being read out to CPU 1, and the combined result is also stored in magnetic disk 20.

(Step 409)

Counter L related to the time phase of the moving image is incremented.

(Step 410)

In (step 407), parameter m of the elapsed time from the reference time of the target image for extraction of a blood vessel image is compared with upper limit value Mn of m in region n thereof. If m is not the same as Mn step 411 is carried out, and if m is the same as Mn step 412 is to proceed.

(Step 411)

Counter m is incremented by 1, and the step moves to step 47.

(Step 412)

Parameter n related to number of the region is compared with Ns obtained in (step 403). If n is not the same as Ns step 413 is to proceed, and if n is the same as Ns the procedure is ended.

(Step 413)

Counter n is incremented, and the step moves to step 405. The moving image of the conclusively synthesized blood vessel image is displayed, for example, on display 18 and stored in magnetic disk 20.

As mentioned above, according to embodiment 1, after simultaneously exciting a plurality of excited planes at a predetermined interval, in order to obtain the signals produced from the blood flowing from the respective excited planes, it is possible to generate the blood vessel image by collecting the echo signals from the excited planes within a minute distance in a minute period of time. Also, influence of turbulence in the phases of the signals can be minimized. The example of the present embodiment also has an advantage of eliminating influence caused by nonuniformity of magnetic fields since the inverting pulses are applied.

### EMBODIMENT 2

Next, an imaging sequence of an MRI method related to embodiment 2 will be described using FIGS. 5 (a) and (b). FIG. 5 (a) is a diagram showing an imaging sequence of the present embodiment, and FIG. 5 (b) is a schematic view showing the correspondence between the excited plane and imaging surface upon executing the imaging sequence related to the present embodiment. The difference of the imaging sequence in FIG. 5 (a) from the imaging sequence in FIG. 2 (a) of embodiment 1 is that there is no application of inverting pulse 203 and gradient magnetic field pulse 204 for slice selection of the imaging surface. As a result, the image generated from NMR signals obtained by gradient magnetic field pulse 205 and 206-1˜206-3 becomes an image wherein the blood vessel image in the imaging space is projected in Gs direction which is as shown in FIG. 5 (b). In the present embodiment also, according to FIG. 5 (b), when images are collected by exciting them using burst RF pulses, the excited planes present stripe pattern on the image data, and the excited planes and the parts that are not excited are arranged alternately.

Embodiment 2 has the same advantage as embodiment 1 to prevent the lowering performance for blood vessel description in the downstream region, and to prevent influence due to turbulence of phases. Also, embodiment 2 has another advantage to save imaging time, since there is no application of inverting pulses.

### EMBODIMENT 3

Next, an imaging sequence of an MRI method related to embodiment 3 will be described referring to FIG. 6. According to the imaging sequence diagram of embodiment 3, burst RF pulses are applied as 201-1 and 201-2 at TR interval. Gradient magnetic field pulses of phase encode are applied as 601-1 and 601-2, and rewound gradient magnetic field pulses are applied as 602-1 and 602-2. Further, at the same time of irradiation of burst RF pulse 201-1 or 201-2, gradient magnetic field pulses 202-1 and 202-2 for slice selection in Gr direction are applied. Echo signals (not shown in the diagram) are collected by applying gradient magnetic field pulses 603-1 and 603-2 for signal readout, and mutually inverting the polar character of gradient magnetic field pulse for slice selection and gradient magnetic field pulse for signal readout. In embodiment 3, signals from blood flowing from the respective excited planes are obtained after a plurality of excited planes are simultaneously excited at a predetermined interval, in the same manner in embodiments 1 and 2. By such method, blood vessel images can be generated by collecting echo signals from the excited planes in a minute period of time and in a minute distance, whereby preventing the lowering performance for blood vessel description in downstream and turbulence of phases.

### EMBODIMENT 4

Next, an imaging sequence of an MRI imaging method related to embodiment 4 will be described using FIG. 7. According to the imaging sequence diagram of embodiment 4, after irradiation of burst RF pulse 501-1, unselected inverting pulses 701-1 and 702-2 are applied at a predetermined time interval, and gradient magnetic field pulses 601-1, 601-2 of phase encode and rewound gradient magnetic field pulses 602-1 and 602-2 are further applied. As in the same manner as embodiments 1˜3, after a plurality of excited planes are simultaneously excited at a predetermined interval, the signals produced from the blood flowing from the respective excited planes are obtained in embodiment 4. By such method, blood vessel images can be generated by collecting echo signals from the excited planes in a minute distance and in a minute period of time, whereby making it possible to prevent the lowering performance for blood vessel description in the downstream part of blood flow and turbulence of the phases. In the present embodiment, gradient magnetic field pulses for slice selection are not applied upon application of inverting pulses, since unselected inverting pulses are applied.

### EMBODIMENT 5

Next, an imaging sequence of an MRI method related to embodiment 5 will be described using FIG. 8. The imaging sequence shown in FIG. 8 is similarly to the one in FIG. 5 (a), but the generation method of image data is different. In the present embodiment, an image is generated based on only the echo signals collected when readout gradient magnetic field is negative (only the echo signals obtained when A/Dm1 and A/Dm2), and the image is also generated based only on the echo signals collected when readout gradient magnetic field is positive (the only echo signals obtained when A/Dp1 and A/Dp2). And the blood vessel image is generated by calculating the difference image of the above-mentioned images.

In the case that echo signals are collected while alternately changing the polar character of readout gradient magnetic field on the negative side and positive side as shown in the imaging sequence of FIG. 8, turbulence of the phases by blood flow changes depending on the polar character. Given this factor, in the present embodiment, the images are reconstructed with respect to each polar character of readout gradient magnetic field, and calculation is performed on the difference in images thereof. By such method, it is possible to extract high quality images of blood vessels.

### EMBODIMENT 6

Next, image composition steps in an MRI method related to embodiment 6 will be described referring to FIG. 9. The only difference of the present embodiment from embodiment 1 is step 408. In step 408 of the present embodiment, all the images used for generation of the moving image at time phase L are summed upon generation of the moving image at time L+1 using the blood vessel image extracted in step 407. The details of the above-mentioned step will be described below. FIG. 9 (a) is a diagram equivalent to FIG. 3 (b) in embodiment 1, and is the image generated after each elapsed time (time 1, time 2, time 3 and time 4) after burst RF pulse 201 is first applied. By these images, it is possible to recognize to what extent the downstream the blood flow is extracted from the excited plane. FIG. 9 (b) shows how the moving image is generated from the image data at each elapsed time (time 1, time 2, time 3 and time 4) obtained in the same manner as FIG. 9 (a).

In the moving image with respect to each time phase, the state of blood flowing from an upstream region along with passage of time is generated. The image is created combining the blood extraction image of the target region in the respective time phases and the blood extraction image in the respective time phases up to that moment. More specifically, first, the moving image is generated at the reference time in time phase 1. Next, region 1 of the moving image at time phase 2 is generated using the blood flow at time 1 in FIG. 9 (b). Next, region 1 of the moving image at time phase 3 is generated summing the blood flow at time 1 and time 2 in FIG. 9 (b). Next, region 1 of the moving image at time phase 4 is generated summing the blood flow at time 1˜time 3 in FIG. 9 (b). Next, region 1 of the moving image at time phase 5 is generated summing the blood flow at time 1˜time 3, and further summing the blood flow of region 2 at time 1. Herein after, the images are generated in the same manner from time phase 6˜time phase 9. The similar moving image as embodiment 1 can be generated using the above-mentioned method.

### EMBODIMENT 7

Next, an MRI method related to embodiment 7 will be described referring to FIGS. 10 (a), (b), and FIGS. 11 (a) and (b). The present embodiment is an imaging method wherein the excited plane is segmented into a plurality of groups arranged alternately to each other, and the respective groups are alternately excited. First, an image-acquisition step in the present embodiment will be described using FIGS. 10 (a) and (b).

The imaging sequence in FIG. 10 (a) is almost the same as FIG. 6 (a), but frequency of burst RF pulses 201-1˜201-3 are made different. More concretely, while burst RF pulses 201-1 and 201-3 has exiting frequency of f0−Δf, burst RF pulse 201-2 has exciting frequency of f0+Δf, and burst RF pulses of two kinds of frequency are alternately applied. And FIG. 10 (b) illustrates how the excited planes excited by two kinds of burst RF pulses are selected. In FIG. 10 (b), excited planes 1001-1 and 1001-3 are excited by burst RF pulses 201-1 and 201-3, and excited planes 1001-2 and 1001-4 are excited by burst RF pulse 201-2. In this way, by alternately applying the burst RF pulses having different frequency, images of the respective regions (the region between excited planes 1001-1 and 1001-2 is set as region 1, the region between excited planes 1001-2 and 1001-3 is set as region 2, the region between excited planes 1001-3 and 1001-4 is set as region 3, and downstream side of excited plane 1001-4 is set as region 4) can be obtained by two times of TR in the case of FIG. 9 (a), whereby enabling extension of recovery time of nuclear-magnetization and improvement of S/N ratio of the blood signals.

Next, image composition step in the present embodiment will be described using FIGS. 11 (a) and (b). FIG. 11 (a) shows, in the present embodiment, the state of blood gradually flowing from the excited plane to downstream, by the images generated from the reference time after each elapsed time, after application of the respective burst RF pulses 201-1˜201-3. According to FIG. 11 (a), while the image of the blood flowing from the excited plane is hardly extracted in the image at the reference time that is immediately after the first application of burst RF pulse, it is recognizable that the extracted region of blood flow in the image extends to downstream as time passes such as time 1, time 2, time 3 and so on. In the image after application of burst RF pulses 201-1 and 201-3 for exciting the excited planes 1001-1 and 1001-3 in FIG. 10 (indicated as excitation 1 in the respective times), the blood vessel images in region 1 and region 3 are extracted. In the image after application of burst RF pulse 201-2 for exciting excited planes 1002-2 and 1001-4 in FIG. 10 (indicated as excitation 2 in the respective times), the blood vessel images in region 2 and region 4 are generated. Given this factor, in the present embodiment, step 401a is inserted between step 401 and step 402 of FIG. 4. Then, by step 401a, the images corresponding to the same time are summed to each other. And the image similar to the one in FIG. 3 (b) can be obtained and combined in the same manner as FIG. 3 (c) using the procedure that follows step 402.

As mentioned above, according to embodiment 7 compared to embodiments 1˜6, since a plurality of excited planes are divided into a number of groups being alternately arranged and they are alternately excited, effective TR is increased upon imaging the respective imaging regions (the regions sandwiched between the excited planes). As a result, S/N ratio of blood signals are improved, since recovery time of nuclear-magnetization can be longer than the cases of embodiments 1˜5.

### EMBODIMENT 8

Next, an MRI method related to embodiment 8 will be described using FIGS. 12 (a) and (b). Embodiment 8 is an example for imaging an object while the object is being transferred, and position of the excited plane is also moved according to movement of the object. FIG. 12 (a) is a diagram showing movement of the blood vessel representing a part of the object and the excited plane thereof, based on the coordinate system of the MRI apparatus being placed quiescently. According to this diagram, both the object and excited plane are moving to the left on the diagram along with the movement of the table.

As shown in FIG. 12 (a), in order to move the position of the excited planes along with movement of the table, an imaging sequence as seen in FIG. 12 (b) is used. More specifically, application frequency of 201-1˜201-3 is increased by Δf to use for slice selection. While application frequency at burst RF pulse 201-1 is F0, application frequency at burst RF pulse 201-2 is f0+Δf, and at burst RF pulse 201-3 application frequency is f0+2Δf. Frequency quantity Δf for increasing at each application of the respective burst RF pulses is calculated based on moving velocity of the table, gradient magnetic field intensity for slice selection and application interval (TR) of burst RF pulses.

By using such imaging sequence, when the object is being moved with the table, the excited plane can be moved along with the movement thereof, and high-quality image of the blood vessel can be extracted.

Also, as for the image composition method, the method illustrated in the flow chart in FIG. 7 can be used in the present embodiment. In other words, in composition of moving images of the present embodiment, since the excited planes move along with the object, those movements can be ignored upon composition of images using the detected echo signals.

### EMBODIMENT 9

Next, an MRI method related to embodiment 9 will be described using FIGS. 13 (a) and (b). Embodiment 9 is an example for imaging while the object is being transferred, the excited planes are made not to move, and the same position in coordinate system viewing from the MRI apparatus is excited. First, FIG. 13 (a) is a diagram showing the movement of the blood vessel representing a part of the object on the basis of coordinate system of an MRI apparatus placed quiescently. In accordance with this diagram, while the object is moving to the left on the diagram along with the movement of the table, the excited plane is not moving along with the movement of the table and is at rest with respect to the MRI apparatus.

In order to perform imaging as illustrated in FIG. 13 (a), an imaging sequence as shown in FIG. 13 (b) is used in the present embodiment. More specifically, application frequency of burst RF pulses 201-1˜201-3 to use for slice selection is set as f and to be constant.

By using such imaging sequence, it is possible to consistently excite the excited plane of the same position with respect to the MRI apparatus.

As for an image composition method, an image composition process considering the relative position with respect to the object at the excited position can be performed using the method as illustrated in FIG. 14 of embodiment 6.

The present invention is not limited to the above-mentioned embodiments, and various changes may be made without departing from the scope of the invention. For example, methods such as spin echo method, high-speed spin echo method, and gradient echo method may be used.

Also, in the above-mentioned embodiment, while the number of excited planes were four in embodiments 1, 2, 6, 7, and three in embodiments 8 and 9, it may be less than three and more than five in the cases such as embodiments 1, 2, 6, 7, and two or more than four in the cases such as embodiments 8 and 9.

While the number for dividing the excited plane into a plurality of groups is set as two in embodiment 7, the number may be more than three. The respective divided groups of the excited plane do not have to be excited alternately, and the echo signals may be obtained by exciting a certain group for a plurality of times, and after that exciting another group for a plurality of times.

Moreover, a method shown in embodiment 7 for imaging by dividing an excited plane into a plurality of groups may be combined with a method as described in embodiments 8 and 9 for imaging while the object is being transferred.

While the blood vessel image on the excited plane is difficult to extract on the image in the above-mentioned embodiment, it is possible to perform interpolation through extracting the blood vessel on the excited plane in downstream by exciting only the excited planes in upstream.

Combination of the image acquisition step and image combination step in the above-mentioned embodiment does not have to be limited to the above-mentioned combination, and other combinations may be applied.

Also, the respective plurality of excited planes does not have to be arranged in parallel, and they may be slightly tilted.

The MRI apparatus used in the present invention includes a program for implementing the above-mentioned MRI methods stored in devices such as magnetic disk 20. The MRI apparatus used in the present invention is also provided with a memory device such as magnetic disk, with information or data generated in the respective process of the above-mentioned MRI method (parameter for executing the imaging sequence, echo signals obtained by the execution of the imaging sequence, image data reconstructed by the echo signals, time-series image data rearranged in (step 701), image data of the moving images in the respective time phases generated in (step 707), and various types of counter for carrying out the flow chart shown in FIG. 7).

Input means is also provided for selectively displaying the generated images or moving images on a device such as display 18. By such means, it is possible for an operator to update the images or moving images displayed on display 18, and to visibly recognize the blood flow with passage of time.

The blood vessel image extracted by the above-mentioned MRI method has a tendency that the pixel value gets larger as getting closer to the excited plane on the upstream side and the pixel value gets smaller as approaching more to downstream, and interpolation may be performed to make it displayed more naturally. In other words, luminance interpolation may be performed to make the pixel value of the blood vessel image on the upstream side of the excited plane small and the pixel value of the blood vessel image on the downstream side large.

The plurality of excitation of the excited plane does not have to be performed simultaneously, and may be sequentially performed from upstream.

Also, the image or moving image from which the excited planes are deleted may be generated, stored, and displayed so that the blood vessel will be clearly visible upon being extracted.

1. A magnetic resonance imaging method comprising:
(1) a step for generating nuclear magnetic resonance by exciting atomic nuclei in a desired region of an object to be examined;
(2) a step for detecting the nuclear magnetic resonance signals generated from the blood; and
(3) a step for extracting a blood vessel image of the object using the detected nuclear magnetic resonance signals,
wherein the desired region excited by the step (1) is a plurality of regions arranged at a predetermined interval.

2. The magnetic resonance imaging method according to claim 1, wherein the plurality of regions are divided into more than two groups, each group executes excitation process in step (1) and detection process in step (2) one time by rotation, and the excitation and detection process in each group by rotation will be repeatedly executed for detection of the nuclear magnetic resonance signals.

3. The magnetic resonance imaging method according to claim 1, wherein the plurality of regions is divided into more than two groups, and each group sequentially executes excitation process in step (1) and detection process in step (2) a plurality of times at a time.

4. The magnetic resonance imaging method according to claim 1, characterized in that, in the step (1), a plurality of excited regions at a predetermined interval are simultaneously excited by applying a burst RF pulse that is a burst high-frequency magnetic field in which a plurality of unit high-frequency magnetic pulses are amplitude-modulated in a form of sinc function and a gradient magnetic pulse for slice selection.

5. The magnetic resonance imaging method according to claim 2, wherein combination of the burst RF pulse which is a burst high-frequency magnetic field in which a plurality of unit high-frequency magnetic field pulses that are amplitude-modulated in a form of sinc function to be applied in the step (1) are arranged at a predetermined interval and the gradient magnetic pulse for slice selection is formed by more than two kinds, and the nuclear magnetic resonance signals are detected by alternately applying the burst RF pulse formed by the respective kinds of combination and gradient magnetic field for slice selection.

6. The magnetic resonance imaging method according to claim 3, characterized in that combination of a burst RF pulse which is a high-frequency magnetic field in a bursting state that are configured at a predetermined interval by a plurality of unit high-frequency magnetic field pulses amplitude-modulated in a form of sinc function to be applied in the step (1) and a gradient magnetic pulse for slice selection is formed by more than two kinds, and a plurality of applications of the burst RF pulse in the respective combinations and gradient magnetic field pulse for slice selection is executed by rotation in each combination.

7. The magnetic resonance imaging method according to claim 1, wherein the step (2) includes a step (4) for detecting nuclear magnetic resonance signals by generating nuclear magnetic resonance phenomenon while changing the polarity of a phase encode gradient magnetic field pulse and the readout gradient magnetic field pulse.

8. The magnetic resonance imaging method according to claim 7, wherein the step (2) includes a step (5) before the step (4) for simultaneously applying a reversing pulse and a gradient magnetic field pulse.

9. The magnetic resonance imaging method according to claim 4, characterized in that:
the burst RF pulse in the step (1) is repeatedly applied at a predetermined interval; and
in the step (2), a phase encode gradient magnetic field pulse, gradient magnetic field pulse for readout and rewind gradient magnetic field pulse are applied in this order, between the adjacent burst RF pulses.

10. The magnetic resonance imaging method according to claim 4, characterized in that, in the step (2), an unselected inverting pulse is repeatedly applied, and a phase encode gradient magnetic field pulse, a readout gradient magnetic field pulse and rewind gradient magnetic field pulse are applied in this order, between the adjacent inverting RF pulses.

11. The magnetic resonance imaging apparatus according to claim 1, wherein the step (3) comprises:
a step (6) for reconstructing images based on the detected nuclear magnetic resonance signals;
a step (7) for arranging the images obtained in the step (6) in time series according to the obtained chronological sequence; and
a step (8) for extracting blood flowing from upstream to downstream as a moving image based on the images arranged in time series by the step (7).

12. The magnetic resonance imaging method according to claim 11, wherein the step (6) comprises:
a step (9) for dividing nuclear magnetic resonance signals into a first nuclear magnetic resonance signal group that are detected while the gradient magnetic field for readout is applied on the positive side and a second nuclear magnetic resonance signal group that are detected while the gradient magnetic field is applied on the negative side;
a step (10) for reconstructing a first image using the first nuclear magnetic resonance signal group, and a second image using the second nuclear magnetic resonance signal group; and
a step (11) for calculating difference between the first image and the second image.

13. The magnetic resonance imaging method according to claim 11, characterized in that the moving image extracted in the step (8) is formed by the plurality of time phases, and the extracted image of the blood is flowing to downstream as the plurality of time phases changes one at a time.

14. The magnetic resonance imaging method according to claim 1, characterized in that the steps (1) and (2) are executed while the object is being transferred, position of the plurality of excited regions are changed according to the moving distance of the object, and nuclear magnetic resonance signals generated by nuclear magnetic resonance phenomenon are detected.

15. The magnetic resonance imaging method according to claim 1, characterized in that the steps (1) and (2) are executed while the object is being transferred, and a blood vessel image is extracted in the step (3) using the positional information indicating where the nuclear magnetic resonance signals obtained in the step (2) are generated from.

16. An magnetic resonance imaging apparatus comprising:
static magnetic field generating means for generating a static magnetic field in an imaging space where an object to be examined is placed;
gradient magnetic field generating means for generating a gradient magnetic field in the imaging space;
high-frequency magnetic field generating means for generating high-frequency magnetic field to induce nuclear magnetic resonance in the object placed in the imaging space;
signal receiving means for detecting nuclear magnetic resonance signals from the object;
signal processing means for reconstructing an image using the detected nuclear magnetic resonance signals;
measurement control means for controlling the gradient magnetic field generating means, high-frequency magnetic field generating means and signal processing means based on a predetermined pulse sequence; and
display means for displaying the image,
wherein the measurement control means controls application of high-frequency magnetic field by the high-frequency magnetic field generating means as to excite a plurality of excited regions arranged at arbitrary intervals in the body of the object.

17. The magnetic resonance imaging apparatus according to claim 16, wherein the signal processing means comprises means for imaging images of blood flowing from the plurality of excited regions with passage of time, as a plurality of images arranged in time series.

18. The magnetic resonance imaging apparatus according to claim 17, characterized in comprising moving image generating means for extracting the blood flow motion as a moving image based on the plurality of images arranged in time series.

19. The magnetic resonance imaging apparatus according to claim 18, characterized in comprising:
a first storage means for storing nuclear magnetic resonance signals detected by the signal receiving means;
a second storage means for storing images reconstructed by the signal processing means; and
a third storage means for storing moving images extracted by the moving image generating means.

20. The magnetic resonance imaging apparatus according to claim 16, wherein the display means displays a plurality of images and moving images, and comprises input means for selecting and displaying the time of the images from time series or the time phase of the moving images.
